(19) United States
(12) Patent Application Publication
(10) Pub. No.: US 2003/0045786 A1
(43) Pub. Date: Mar. 6, 2003

(54) Method and apparatus for analysis of structures

(75) Inventors: Yong D. Zhao, Plymouth, MN (US); Cathleen J. McMahon, Coon Rapids, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 09/946,705

(22) Filed: Sep. 5, 2001

(51) Int. Cl. A61B 5/04

(52) U.S. Cl. 600/372

(57) ABSTRACT

A method and an apparatus for performing mechanical and structure analysis for medical implant systems. The structure to be analyzed is defined. A structure model of the structure is developed. A physical structure analysis is performed upon the structure. An integrating structure analysis based upon the structure model and the physical structure is performed, the integrating structure analysis to generate data for performing at least one of a design, development, simulation, safety evaluation, and manufacturing of the structure.

### BACKGROUND OF THE INVENTION

1. Field of the Invention

This invention relates generally to implantable medical devices, and more particularly, to a method and apparatus for performing efficient analysis of structures relating to implantable medical devices.

2. Description of the Related Art

The technology explosion in the implantable medical devices industry has resulted in many new and innovative devices and methods for analyzing and improving the health of a patient. The class of implantable medical devices now includes pacemakers, implantable cardioverters, defibrillators, neural stimulators, and drug administering devices, implantable leads, implantable probes, among others. Today's state-of-the-art implantable medical devices are vastly more sophisticated and complex than early ones, capable of performing significantly more complex tasks. The therapeutic benefits of such devices have been well proven.

There are many implementations of implantable medical devices that provide data acquisition of important physiological data from a human body. Many implantable medical devices are used for cardiac monitoring and therapy. Often these devices comprise sensors that are placed in blood vessels and/or chambers of the heart. Often these devices are operatively coupled with implantable monitors and therapy delivery devices. For example, such cardiac systems include implantable heart monitors and therapy delivery devices, such as pace makers, cardioverter, defibrillators, heart pumps, cardiomyostimulators, ischemia treatment devices, drug delivery devices, and other heart therapy devices. Most of these cardiac systems include electrodes for sensing and gain amplifiers for recording and/or driving sense event signals from the inter-cardiac or remote electrogram (EGM).

As the functional sophistication and complexity of implantable medical device systems have increased over the years, it has become increasingly useful to include a system for facilitating communication between one implanted device and another implanted or external device, for example, a programming console, monitoring system, or the like. Shortly after the introduction of the earliest pacemakers, it became apparent that it would be desirable for physicians to non-invasively obtain information regarding the operational status of the implanted device, and/or to exercise at least some control over the device, e.g., to turn the device on or off or adjust the pacing rate, after implant. As new, more advanced features have been incorporated into implantable devices, it has been increasingly useful to convey correspondingly more information to/from the device relating to the selection and control of those features.

Additionally, for diagnostic purposes, it is desirable for the implanted device to be able to communicate information regarding the device's operational status and the patient's condition to the physician or clinician. In fact, a wide variety of data may be collected by the implanted device and provided to the physician or clinician. The data provided by the implanted device may be real-time or recorded data. For example, implantable devices are available that can transmit a digitized electrical signal reflecting electrical cardiac activity (e.g., an ECG, EGM or the like) for display, storage, and/or analysis by an external device. In addition, known pacemaker systems have been provided with what is referred to as Marker Channel™ functionality, in which information regarding the pacemaker's operation and the occurrence of physiological events is communicated to an external programming unit. The Marker Channel™ information can then be printed or displayed in relation to an ECG so as to provide supplemental information regarding pacemaker operation. For example, events such as pacing or sensing of natural heartbeats are recorded with a mark indicating the time of the event relative to the ECG. This is helpful to the physician in interpreting the ECG, and in verifying proper operation of the pacemaker. One example of a Marker Channel™ system is disclosed in U.S. Pat. No. 4,374,382 to Markowitz, entitled “Marker Channel™ Telemetry System for a Medical Device.” The Markowitz '382 patent is hereby incorporated by reference herein in its entirety.

Generally, a number of physiological data such as ventricular pressure, oxygen supply in the patient's blood, EGM data, and the like, are collected and stored by data acquisition devices implanted into a human body. Collecting these sets of physiological data set often require intricate probing devices such as leads, probes, wires, and the like. These probing devices are generally designed to be slender structures with desired flexibility and reliability. Much analysis is needed in designing and creating these probing devices.

Implantable medical devices, such as cardiac pacing and defibrillation leads, neurological leads, neurological catheters, cardiac catheters, and lead delivery systems, consist of multiple components that are slender. These components include various coils, cables, insulation tubing, adhesives, electrodes, pull wires, and the like. The cross section of the components may be coaxial or non-coaxial, symmetrical or unsymmetrical, single lumen or multilumen, with or without clearances between the components. Many calculations for mechanical and structures analysis are performed for design and/or safety evaluation including various operation factors such as interaction between leads and lead delivery systems, interaction between the lead components, and interaction between device and the heart or cardiac veins, and the like.

Analyzing many of the operation factors experienced by the implanted device can become very cumbersome and inefficient. Product modeling and behavior modeling can become very calculation-intensive and require an inordinate amount of computing resources. Many times, such analysis can also become time-consuming, or even impossible, thereby delaying delivery of new and innovative products.

The present invention is directed to overcoming, or at least reducing the effects of, one or more of the problems set forth above.

### SUMMARY OF THE INVENTION

In one aspect of the present invention, a method is provided for performing structural analysis. The structure to be analyzed is defined. A structure model of the structure is developed. A physical structure analysis is performed upon the structure. An integrating structure analysis based upon the structure model and the physical structure is performed, the integrating structure analysis to generate data for performing at least one of a design, development, simulation, safety evaluation, and manufacturing of the structure.

In another aspect of the present invention, a system is provided for performing structural analysis. The system of the present invention comprises: means for defining the structure to be analyzed; means for developing a structure model of the structure, developing the structure model comprising generating a part finite element analysis (FEA) model based upon a physical test model and an environmental effect; means for performing a physical structure analysis upon the structure, performing the physical structure analysis comprising sending an experimental stiffness curve to a global finite elements analysis (FEA) model, the experimental stiffness curve being based upon a physical test performed on the structure; and means for performing an integrating structure analysis based upon the structure model and the physical structure, the integrating structure analysis to generate data for performing at least one of a design, development, simulation, safety evaluation, and manufacturing of the structure based upon the global finite elements analysis (FEA) model generated from the physical test data and the part finite elements analysis (FEA) model.

### BRIEF DESCRIPTION OF THE DRAWINGS

The invention may be understood by reference to the following description taken in conjunction with the accompanying drawings, in which like reference numerals identify like elements, and in which:

FIG. 1A is a simplified diagram of an implementation of an implantable medical device in accordance with one illustrative embodiment of the present invention;

FIG. 1B illustrates an interaction between a sensor and the implantable medical device of FIG. 1, in accordance with one illustrative embodiment of the present invention;

FIG. 2 illustrates a simplified block diagram representation of a system for implementing the principles of the present invention, in accordance with one illustrative embodiment of the present invention; and

FIG. 3 illustrates a flowchart depiction of a method performing an efficient mechanical and structural analysis, in accordance with one illustrative embodiment of the present invention;

FIG. 4 illustrates a flowchart depiction of a method of developing a structure model, as indicated in FIG. 3, in accordance with one illustrative embodiment of the present invention;

FIGS. 5A-5G illustrate diagrams that represent load modes in the direction of interest of analysis of structures and components for modeling and other analysis, in accordance with one embodiment of the present invention;

FIG. 6 illustrates a flowchart depiction of a method of performing a mechanical and structural analysis, as indicated in FIG. 3, in accordance with one illustrative embodiment of the present invention; and

FIG. 7 illustrates a flowchart depiction of a method of performing an integrating structure analysis, as indicated in FIG. 3, in accordance with one illustrative embodiment of the present invention.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF SPECIFIC EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

There are many discrete processes involving collecting, storing, and presenting physiological trends of a patient. Generally, an inordinate amount of analysis is performed on components used as implantable medical devices. Implantable medical devices, such as cardiac pacing and defibrillation leads, neurological leads, neurological catheters, and lead delivery systems, consist of multiple components that may be designed under extensive analysis for proper operation. Embodiments of the present invention provide for utilizing simplified models of structures or components used as implantable medical devices, and performing complex analysis using the models.

Embodiments of the present invention provide a generic method for calibrating the composite structural stiffness and material and mechanical properties of a real device or a composite structure to a simple structural model such as a solid cylinder, tube, beam, etc. Embodiments of the present invention provides for simplifying components and/or structures in an accurate fashion. Methods taught by embodiments of the present invention facilitate performing an effective numerical analysis of a complex structure that may be difficult, or even impossible, otherwise. In one embodiment, the composite structural stiffness of the real structure, in a direction of interest, may be obtained by measuring a single or combined tension (relating to one or a plurality of vectors), compression, bending, and/or torsional load(s) applied, and measuring the corresponding resultant displacement at the location of interest.

In one embodiment, a stiffness test performed on a simplified structure may be a displacement-controlled test. In an alternative embodiment, the test performed on a simplified structure may be a loading-controlled test. Results from the tests may be used to generate stiffness curves, which in one embodiment, can be plotted as a displacement versus reaction, loading force, or moment, diagram. A particular plotted stiffness curve, as obtained by a physical test, then may be then recreated in a component finite element analysis (FEA) model, using substantially the same test boundary conditions, load, and gage length. In one embodiment, those skilled in the art having the benefit of the present disclosure can perform the FEA analysis.

The actual component (structure), or a sample of the actual component, under analysis (which can be a simplified structure representing a more complex component) used in the test, may be represented as a simplified structure that retains either the actual inner or outer diameter, for maintaining a predetermined physical clearance between the component of the implantable medical devices under analysis. The mechanical material properties in terms of a stress-strain relationship relating to the simple structure can be approximated as a linear, a bilinear, a piecewise linear material, or the like for the purpose of analysis. The approximation of the simple structure as a linear, bilinear, a piecewise linear material, or the like, may be based upon the shape of the test stiffness curve described above.

In one embodiment, the artificial material properties, such as the Young's modulus, and either the outer diameter or the inner diameter of the simplified component structure in the FEA model, are adjusted iteratively. These adjustments, or calibrations, are performed until the FEA-generated stiffness curve substantially matches the stiffness curve measured experimentally for the real device or component being analyzed. This calibrated, simplified model can then be used more effectively and accurately for various numerical analyses or analytical calculations. When the method described above is used to calibrate the artificial material properties, such as the Young's modulus of a small-sized wire, cable, or coil, which is generally difficult to obtain using experimental testing, the FEA model may contain substantially similar geometries, gage length, loading, and boundaries as compared to the physical test(s). In one embodiment, the Young's modulus is the only variable in the FEA model and is calculated such that the FEA model produces a stiffness curve that is substantially similar to the stiffness curves produced from the physical test(s). The Young's modulus thus determined comprises the residual stress/strain effects introduced in the wire/coil manufacturing process, which is generally more realistic and accurate as compared to the data tested from raw material or large-sized specimens.

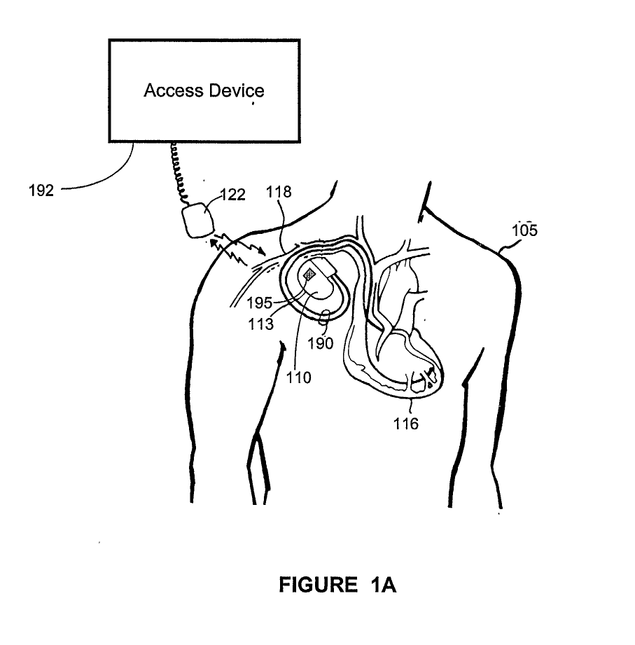

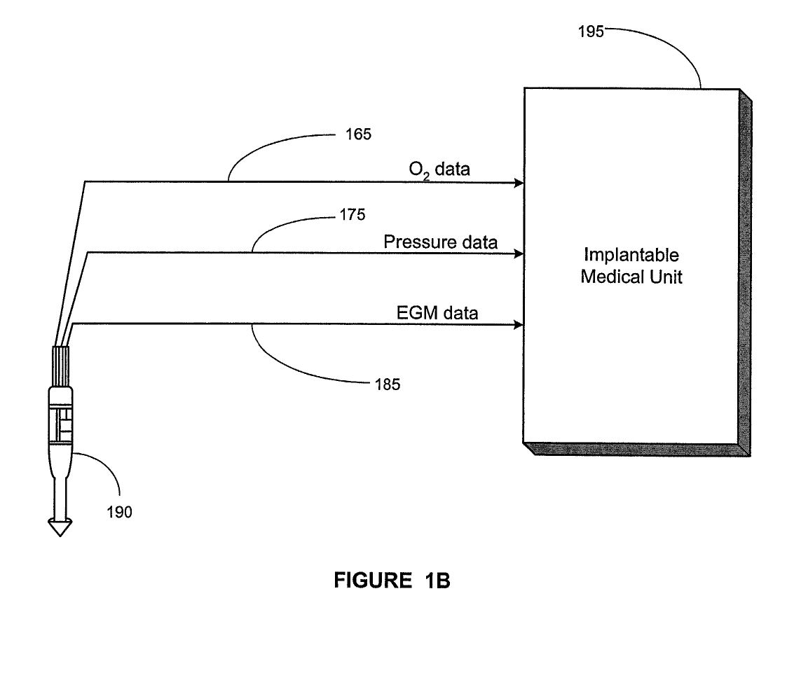

Although principles of the present invention can be utilized in a number of applications involving mechanical and structure analyses, embodiments of the present invention is presented in the realm of implantable medical systems for ease and clarity as to not obscure the fundamentals of the present invention. FIG. 1A illustrates one embodiment of implementing an implantable medical system to implant an implantable medical device into a human body. A sensor 190 (e.g., leads) placed upon the heart 120 of the human body 105 is used to acquire and process physiological data. An implantable medical unit 195 collects and processes a plurality of data acquired from the human body. In one embodiment, the implantable medical unit 195 may be implemented in a pacemaker 110. The data acquired by the implantable medical unit 195 can be monitored by an external system, such as the access device 192 comprising a programming head 122, which remotely communicates with the implantable medical unit 195. The programming head 122 is utilized in accordance with medical device programming systems, for facilitating two-way communication between the pacemaker 110 and the access device 190.

In one embodiment, a plurality of access devices such as the sensor 190 can be employed to collect a plurality of data processed by the implantable medical unit 195 in accordance with embodiments of the present invention. The pacemaker 110 is housed within a hermetically sealed, biologically inert outer canister or housing 113, which may itself be conductive so as to serve as an electrode in the pacemaker's pacing/sensing circuit. One or more pacemaker sensors/leads, collectively identified with reference numeral 190 in FIG. 1A are electrically coupled to the pacemaker 110 in a conventional manner and extend into the patient's heart 116 via a vein 118. Disposed generally near a distal end of the sensors 190 are one or more exposed conductive electrodes for receiving electrical cardiac signals or delivering electrical pacing stimuli to the heart 116. The sensors 190 may be implanted with their distal end situated in either the atrium or ventricle of the heart 116. In an alternative embodiment, the sensors 190, or the leads associated with the sensors 190, may be situated in a blood vessel on the heart 116, such as a vein 118. Proper analysis of the components used in conjunction with the implantable medical device is relevant for effective design, production, simulation, safety evaluation, and operation of the implantable medical system illustrated in FIG. 1A.

Turning now to FIG. 1B, the implantable medical unit 195 receives physiological data from the sensor 190. The sensor 190 is implanted into the body of a patient 105 such that the desired physiological data can be acquired. In one embodiment, the sensor 190 provides O₂ data, which can indicate the amount of oxygen in a patient's blood. The implantable medical unit 195 also receives the ventricular pressure data of the patient from the sensor 190. Furthermore, the implantable medical unit 195 receives cardiac electrogram signals (EGM) from the sensor 190. The O₂ data, the pressure data, and the EGM are presented to the implantable medical unit 195 via lines 265, 275, and 285, respectively. The flexibility, durability, and conformity of the sensor 190 is important in the proper operation of the implantable medical system illustrated in FIG. 1A. Embodiments of the present invention can be utilized to design, develop, simulate, evaluate safety (e.g., device structural integrity, such as fatigue analysis), and manufacture reliable, efficient, and durable sensors 190 and components associated with the sensors 190. Furthermore, embodiments of the present invention can be used to perform a device-anatomy interaction, such as simulating the implantable medical device 220 while interfacing with a portion of the anatomy of a patient.

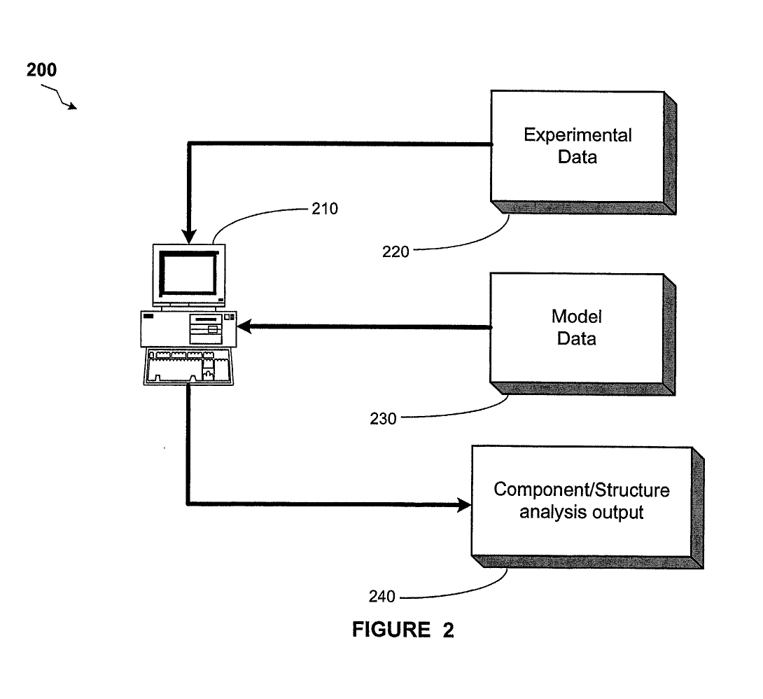

Turning now to FIG. 2, a simplified block diagram of a system 200 for performing the mechanical and structure analysis in accordance with one illustrative embodiment of the present invention is illustrated. A computer system 210 receives experimental data 220 relating to a component or a structure under analysis. In one embodiment, experimental data based on physical tests, which includes physical stiffness tests, described above is sent to the computer system 210. The computer system 210 also receives model data 230 based upon modeling analysis of the component or structure, as described above. In one embodiment, the model data 230 may be generated within the computer system 210. Using the principles taught by embodiments of the present invention, described above and below, the computer system 210 generates a component/structure analysis output 240. In one embodiment, the computer system 210 may perform a simulation of the operation of a component/structure based upon the experimental data 220 and the model data 230. The component/structure analysis output can be utilized to design, develop, simulate, evaluate safety, and/or manufacture components or structures for the medical implant system illustrated in FIG. 1A.

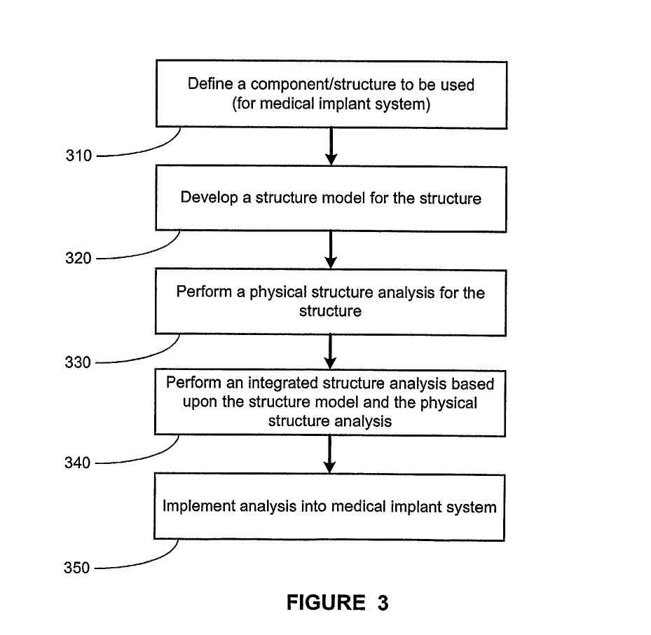

Turning now to FIG. 3, a flowchart depiction of a method for performing structure analysis in accordance with one embodiment of the present invention is illustrated. In one embodiment, the structure to be used in conjunction with the implantable medical system is defined (block 310). Generally, the structure to be defined is a physical structure that is to be placed inside the human body. The defined structure can be a lead, a catheter, a lead delivery system, coils attached to a lead, a cable, an insulation tubing, a structure that may adhere a lead end onto a portion of the body, electrodes, pull wires, and the like.

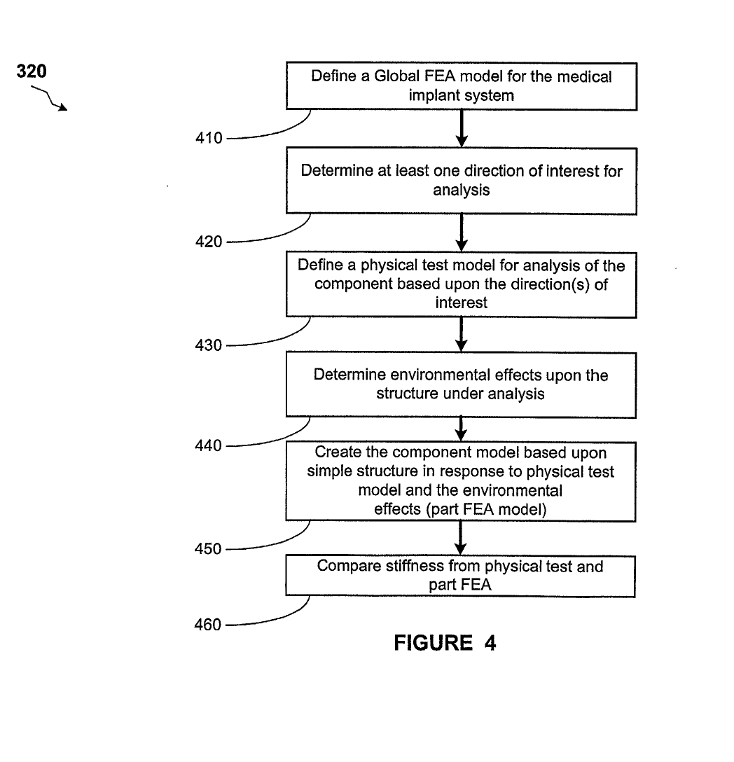

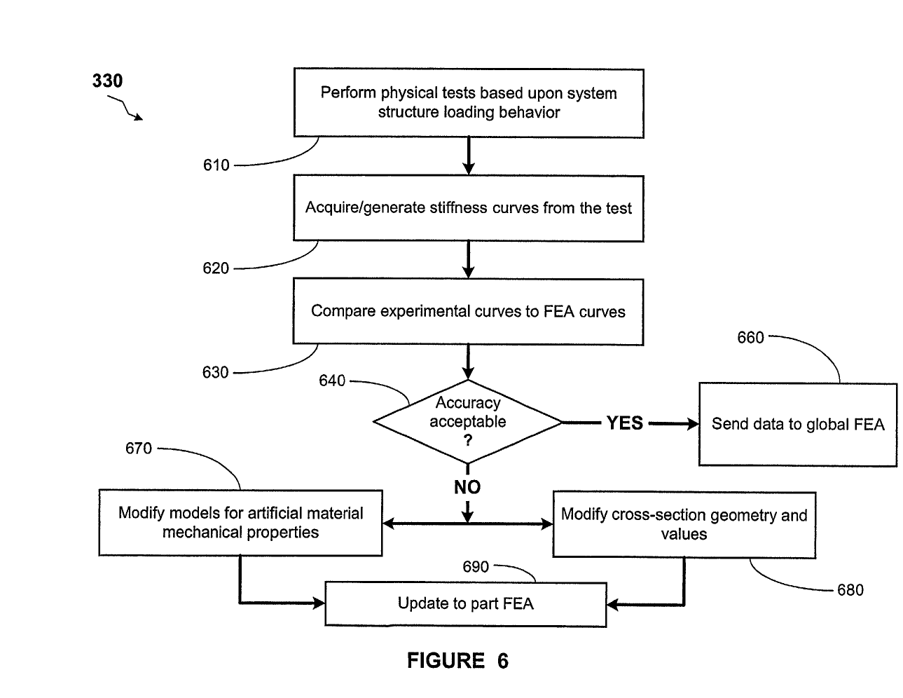

Once a structure is defined, a structure model for the structure/component is developed (block 320). In one embodiment, developing a structure model comprises developing a simplified component model of a more complex component. In an alternative embodiment, developing a structure model comprises developing a mathematical model of a component. A more detailed description of developing a structure model is provided in FIG. 4 and accompanying descriptions below. Once a structure model is developed, a physical structure analysis, which includes a physical stiffness test of a component, is performed in conjunction with the structure model (block 330). A more detailed description and illustration of performing the physical description analysis indicated in block 330, is provided in FIG. 6 and the accompanying descriptions below.

Once the structure model is developed and a component calibration using the physical structure analysis is performed, an integrating structure analysis is performed (block 340). In one embodiment, the integrating structure analysis is based upon the simplified component model with real stiffness from the physical tests. The integrating structure analysis comprises comparing results from the physical structure analysis (e.g., stiffness results) and the component structure model to develop behavior of a particular structure under certain load conditions. The integrating structure analysis comprises a stiffness calibration analysis, which includes a suitable material model. The integrating structure analysis also comprises a global finite element analysis (FEA) in order to analyze the entire structure. The integrating structure analysis may comprise performing a numerical analysis, or in an alternative embodiment, performing a closed-form analysis. The integrating structure analysis comprises developing a simplified model and performing analysis to study the structure of a more complex structure. Once the integrating structure analysis is performed, as indicated in block 340, the structure is then implemented into the implantable medical system (block 350). Implementing a structure into the implantable medical system comprises developing a physical structure such as a lead and integrating the lead with an implantable medical device and inserting the lead into the human body.

Turning now to FIG. 4, a more detailed flowchart depiction of developing a structure model as indicated in block 320 of FIG. 3 is illustrated. In one embodiment a global FEA is defined for analysis of a particular structure to be analyzed (block 410). The system 200 reviews the geometrical structure of a component under analysis. The system 200 also determines the deforming behaviors of the component under service loads and environmental effects for an assembled product or medical implant system that consists of several parts of components or subsystems. The global FEA model comprises analysis of simple structures (e.g., beam, tube, bar, etc.) with appropriate clearances between the plurality of parts for all parts or subsystems for stiffness analysis of the whole product or medical implant system. The global FEA model also comprises analysis of simple structures with appropriate clearances for all parts of subsystems except one part with real geometries for stress analysis for that individual part. In this case, the simple structure includes a beam, tube, bar, etc., wherein one solid part with specific cross-section shape and dimension consistent with stiffness-orientation is analyzed.

Once a global FEA model is defined, a direction of interest for analysis of a particular structure is determined (block 420). In one embodiment, the direction of interest is a combined lateral force direction. The composite structural stiffness of the real structure, in the direction of interest, can be obtained by measuring a single or combined tension, compression, bending, and/or torsional loads applied and recording the resultant displacement (i.e., a physical test). In embodiment, the test may be a displacement-controlled physical test. In an alternative embodiment, the test may be a loading-controlled physical test.

Diagrams from the resultant data from the test(s), such the stiffness curves resulting from the approach described above, can be plotted. Such a plot may be represented by a diagram that illustrates a curve based upon the displacement versus the reaction, loading force, or the moment. One such plot that provides a deflection curve plotted with displacement versus reaction force is provided in Appendix A.

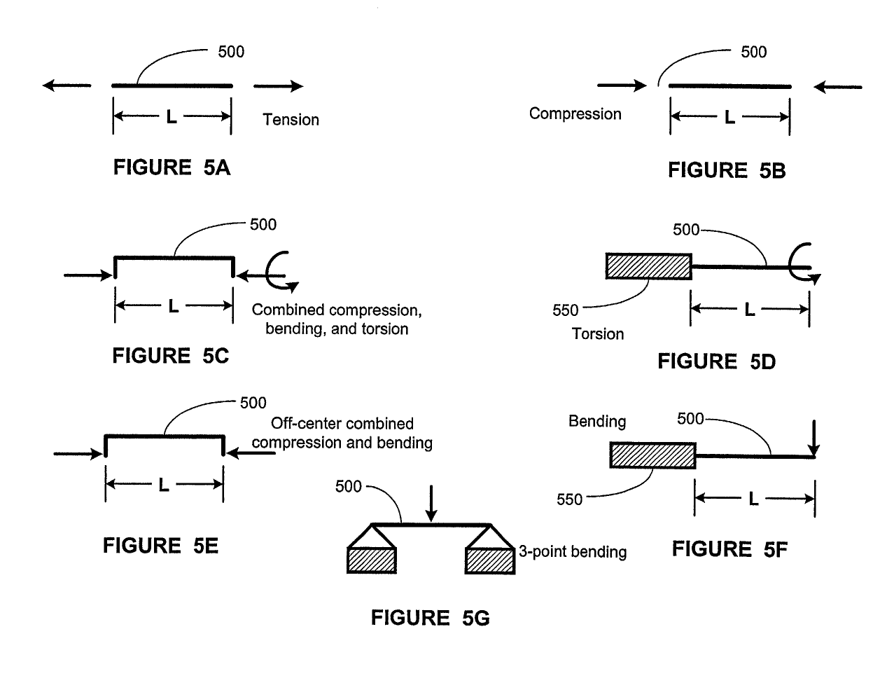

A plurality of load types of interest for a particular structure is illustrated in FIGS. 5A through 5G. FIG. 5A illustrates a tension model, which provides a tension force in the opposite direction of a structure 500 that has a gage length, L. FIG. 5B illustrates a compression model wherein force is applied in a compressive direction upon a structure 500 that has the length L. The compression model provides analysis of buckling and post-buckling studies. FIG. 5C illustrates a structure 500 where the direction of interest of analysis is a combination of compression-bending-torsion analyses. This analysis provides an insight of the behavior of a lead 114 that is traversed through an intricate system of veins on the left side of a human heart.

FIG. 5D illustrates a structure attached to a fixed point 550 undergoing a torsion moment, M. FIG. 5E illustrates a load of interest analysis on a structure 500 undergoing an off-center compression-bend combined model. This analysis provides an insight of the forces that a lead 114 would experience during an implant push process. FIG. 5F illustrates a load of interest analysis of a structure 500 undergoing a cantilever bending analysis, wherein a force is applied at one end of the structure 500 and the opposite end of the structure at a distance L is positioned upon a fixed object 550. FIG. 5G illustrates a load of interest analysis wherein the structure 500 undergoes a modified 3-point bend analysis. The modified 3-point bend analysis comprises a fixation at one end of the structure 500 and a force applied on the opposite end at a distance L of the structure, and a force applied at a point L, upon the structure 500. The analyses performed on the structure 500 can be analyzed as a simplified structure, such as a cylinder, which provides for more efficient development of an eight nodal element for finer mesh structure analysis.

Turning back to FIG. 4, once the load and direction of interest for analysis of a structure 500 is performed, a physical test model is defined (block 430). In one embodiment the physical test model is used to formulate a model for physically testing the structure 500. The physical test model is generated to measure the composite stiffness curves in terms of load and displacement at locations/directions of interest for each part or subsystem (e.g., component/structure) of a medical implant system. In one embodiment, the composite stiffness curves are formulated based upon single or combined loads of tension, compression, bending, and torsion. The physical test model comprises the orientation of part/structure 500 geometry, the load type, the load path, the boundary conditions, and the gage length. Furthermore, environmental effects (e.g., degradation, aging, erosion or corrosion, fluid effects, temperature, creep, etc.) that would be felt by the structure 500 being analyzed may be included in the test sample preparation or in the test process. Once the physical test model is defined, a determination is made regarding the environmental effects felt by the structure (block 440).

Once a physical test model is completed and corresponding environmental effects have been considered, a part FEA model based upon simple structure in response to the direction of interest, the physical test model, and environmental effects, is created (block 450). The part FEA model may be created for a plurality of components that may effect the integrity of the structure 500, including tension, torsion, stiffness, and the like. In one embodiment, the part FEA model is created to calibrate the composite structure stiffness obtained from the physical test of the part or subsystem.

In embodiment, the part FEA model is provided substantially the same load type and path, boundaries, gage length, cross section geometries, with the exception of one geometry parameter for use as a given variable. The given variable may be either the inner diameter or the outer diameter of the structure under analysis, depending on its contact interaction with other parts/structures in the subsystem of the medical implant system. The inner diameter and the outer diameter of the structure under analysis are generally calculated to maintain a real physical clearance between components or structures in the global FEA model.

In one embodiment, the part FEA model is assigned a specific material model to perform parametric FEA computations, such that the iterated material model constants will result a structure stiffness curve that is similar to the structure stiffness curve generated from the physical test (i.e., stiffness calibration). The material models for the stiffness calibration may be isotropic or anisotropic. Furthermore, the material models for the stiffness calibration may be linear or nonlinear. The material elasticity may be linear elasticity (Hooke's law) or hyperelasticity (in terms of Arruda-Boyce, Mooney-Rivin, Neo-Hookean, Ogden, Polynomial, and other material models). The material plasticity may be rated independent or dependent (e.g., elasticplastic, bilinear, pice-wise linear, power law, etc.). Consequently, the simplified part/structure with the calibrated geometries and artificial material mechanical properties can then represent the more complicated, actual part or subsystem, since they have substantially similar composite structure stiffness under substantially similar loading in the direction of interest.

In one embodiment, after the completion of the physical test model, the environmental effects analysis, and the part FEA model, a physical interaction curve, such as the stiffness curves described above, is generated (block 460). In one embodiment, stiffness curves using results from the physical test and the part FEA is generated. One example of a curve that provides an indication comparison of the physical test versus the FEA, which plots a pushing displacement versus a pushing reaction force, is illustrated in Appendix B. The completion of the blocks illustrated in FIG. 4 substantially completes the step of developing a structure model as indicated in block 320 of FIG. 3.

Turning now to FIG. 6, a flowchart depiction of one embodiment of performing the integrating structural analysis indicated in block 330 of FIG. 3 is illustrated. One or a plurality of physical tests based upon the physical test modeling is performed (block 610), which comprises performing physical tests based upon system structure loading behavior for each component under analysis. The physical test performed on a structure 500 may comprise testing a plurality of components. In one embodiment, the physical test comprises performing the various tests in the direction of interest as indicated in FIG. 5. One example of performing a physical test for a bending load test upon a lead is illustrated in Appendix C.

One or more components may be tested on a particular structure 500. Results from the physical tests may be used to acquire and/or generate physical displacement curves, such as stiffness curves (block 620). In one embodiment, a targeted curve is generated based upon FEA results. The experimental stiffness curves are then compared to the target curves to determine the amount of deviation of physical curves (block 630). A determination is made whether the stiffness curve represents an accuracy that is acceptable within a predetermined margin of error (block 630).

When a determination is made that the stiffness curve accuracy is acceptable based upon a predetermined range of errors, the physical test calibrated component data (simplified component) is sent to the global FEA (block 660). The global FEA model then uses the data from the physical experiments/tests calibration to perform structural analysis. When a determination is made that the stiffness curve accuracy is not within an acceptable margin of error, models for artificial material mechanical properties are modified (block 670), whose output are then sent to the simplified (part) FEA model for analysis. Furthermore, cross-section geometry shape and values are modified for the structure 500 being analyzed (block 680). The models for artificial material mechanical properties and the cross-section geometry shapes and values used to update the simplified (part) FEA model s(block 690). Therefore, the modeling system gains insightful data represented by simplified physical tests of structures 500 of interest. The completion of the steps indicated in FIG. 6 substantially completes the process of performing physical structure analysis as indicated in block 330 of FIG. 3.

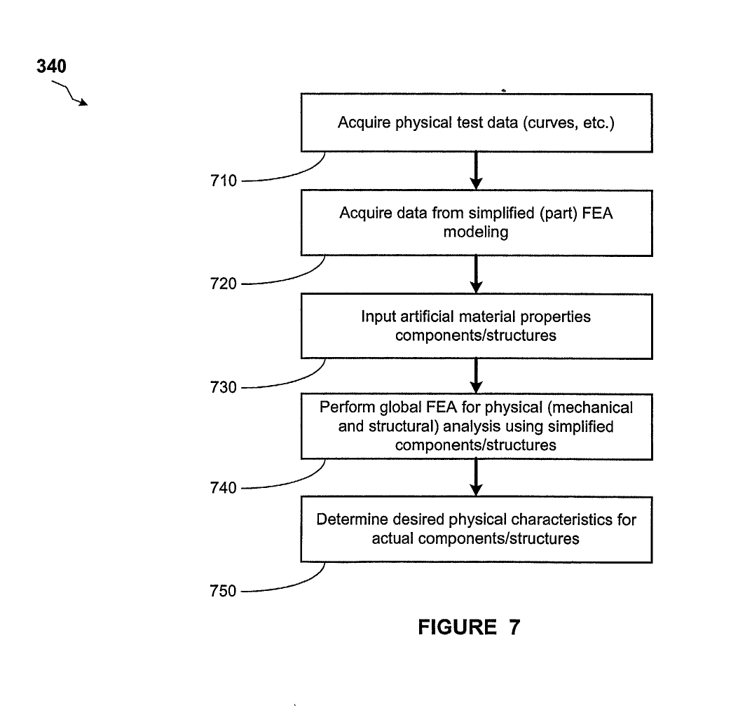

Turning now to FIG. 7, a flowchart depiction of one embodiment of performing an integrated structural analysis, as indicated in block 340 of FIG. 3, is illustrated. The physical (stiffness) test data generated during the physical structural analysis is acquired (block 710). The physical test data comprises results from physical experiments on the structure 500, physical experiments performed on the simplified structure, curves representing displacement versus force, etc. Furthermore, data from the simplified/part FEA modeling is acquired (block 720), for maintaining realistic physical contact interaction between components. Data from the simplified/part FEA modeling comprises analysis of structures 500 based upon calculations for a plurality of factors such as stiffness, lateral force, etc. In one embodiment, the artificial material properties, such as the Young's modulus, are determined for one or more components of the structure 500 (block 730).

Using the physical test data and the data from the simplified/part FEA modeling, Young's modulus, Ogden model constants, and the like, a global FEA is performed for executing a physical (mechanical and structural) structure/stress analysis, using simplified components (block 740). This process may comprise calibrating material and mechanical properties like Young's modulus yielding stress, elasticity, etc. for a finished component/structure under analysis. A global FEA may comprise analyzing a plurality of models and geometries for an individual part of subsystems calibrated by the part FEA model and the physical test model.

In one embodiment, the global FEA modeling is used to simulate the interaction between a lead 114, lead delivery system, and the heart or cardiac veins during implant or explant of a component of a medical implant system. The global FEA modeling can also be used to predict stress and strain that may be subjected upon the device/structure under analysis, facilitating “useful life” calculations. The global FEA modeling can also be used to evaluate structural performance for developing product design concepts, identifying device implant or explant procedures, and improving quality and reliability of a device to be used in conjunction with the medical implant system.

However, the slender device and components (which exhibit varied material and mechanical properties) are sufficiently structurally complex that a large number of elements are generally created to yield the mesh quality for performing an accurate analysis. Furthermore, a large number of elements are often required to include the physical interaction of contact between components/structures when deformed. The resulting model is often very large and computationally expensive such that yielding useful results in a timely fashion or may be difficult. Embodiments of the present invention can be used to significantly reduce the number of elements in the global FEA model so that it can be analyzed more efficiently while maintaining the realistic structural stiffness and contact interaction of the component/structures in the model. When a single component/structure is of primary interest, that component/structure is modeled using realistic geometries, dimensions, and material properties, while the remaining components/structures are secondary in the global FEA model and may be simplified using the inventive methods provided herein.

Data from the global FEA model is then used to determine the desired physical characteristics for actual components/structures to be used in conjunction with the implantable medical system (block 750). The completion of steps indicated in FIG. 7 substantially completes the process of performing the structural analysis indicated in block 340 of FIG. 3. The results are then used to implement into designing, developing, simulating, evaluating safety (e.g., device structural integrity, such as fatigue analysis), and/or manufacturing components/structures that comprises structures used in conjunction with an implantable medical system. In one embodiment, the computer system 210 may perform simulation of components, structures, and materials using the principles described above. The principles taught by embodiments of the present invention, can be utilized to perform physical and structural analysis on a plurality of materials used in a wide range of applications. Data provided by the implementation of the embodiments of the present invention can be used to perform a variety of simulation, generally performed before physical implementation of the structures that are simulated.

The above detailed description is an illustrative example of an embodiment in accordance with the present invention, of the implementation of an implantable medical system described above. It should be appreciated that other implementations and/or embodiments can be employed within the spirit of the present invention. The teachings of the present invention can be utilized for a variety of systems where structural analysis would be beneficial.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

[image]

[image]

[image]

What is claimed:

1. A method for analyzing a structure, comprising:
defining said structure to be analyzed;
developing a structure model of said structure;
performing a physical structure analysis upon said structure; and
performing an integrating structure analysis based upon said structure model and said physical structure, said integrating structure analysis to generate data for performing at least one of a design, development, simulation, safety evaluation, and manufacturing of said structure.

2. The method described in claim 1, further comprising implementing said structure into a medical implant system based upon said integrating structure analysis.

3. The method described in claim 1, further comprising performing a device-anatomy interaction simulation.

4. The method described in claim 1, wherein performing a physical structure analysis upon said structure further comprises performing a physical stiffness test upon said structure.

5. The method described in claim 1, wherein defining said structure to be analyzed farther comprises defining at least one of a cardiac pacing lead, a defibrillation lead, a neurological lead, a neurological catheter, a cardiac catheter, and a lead delivery system.

6. The method described in claim 1, wherein developing a structure model of said structure further comprises:
determining a load in a direction of interest for analysis of said structure;
defining a physical stiffness test model based upon said direction of interest of analysis;
evaluating at least one environmental effect upon said structure;
generating a part finite element analysis (FEA) model based upon said physical test model and said environmental effect; and
calibrating a stiffness curve based upon said part finite element analysis (FEA) model.

7. The method described in claim 6, wherein determining the load in the direction of interest for analysis of said structure further comprises determining one
a load of compression force upon said structure;
a load of bending force upon said structure;
a load of torsion force upon said structure;
a load of compression-bending-torsion force upon said structure;
a load of compression-bending force upon said structure; and
a load of three-point bending force upon said structure.

8. The method described in claim 6, wherein determining at least one environmental effect upon said structure further comprises determining at least one
a degradation factor upon said structure;
an aging factor upon said structure;
an erosion factor upon said structure;
a corrosion factor upon said structure;
a temperature effect factor upon said structure; and
a fluid effect factor upon said structure.

9. The method described in claim 6, wherein generating a part finite element analysis (FEA) model based upon said physical test model and said environmental effect further comprises calibrating a composite structure stiffness for said structure.

10. The method described in claim 6, wherein calibrating a stiffness curve based upon said part finite element analysis (FEA) model further comprises generating a computed stiffness curve.

11. The method described in claim 10, wherein performing a physical structure analysis upon said structure further comprises:
performing a physical test of said structure based upon a physical modeling;
generating an experimental stiffness curve based upon said physical test;
comparing said experimental stiffness curve to a target curve from said part finite element analysis (FEA) to generate comparison data;
determining whether said comparison data is inside a predetermined range;
sending said experimental stiffness curve to a global finite elements analysis (FEA) in response to a determination that said comparison data is inside a predetermined range; and
sending said experimental stiffness curve to said part finite elements analysis (FEA) in response to a determination that said comparison data is inside a predetermined range.

12. The method described in claim 6, wherein performing an integrating structure analysis based upon said structure model and said physical structure
acquiring physical test data based upon said physical structure analysis;
acquiring data from said part finite element analysis (FEA);
generating a global finite element analysis (FEA) model based upon said physical test data and said data from said part finite element analysis (FEA); and
determining a desired physical interaction characteristic for said structure.

13. A method for analyzing a structure for implementation in a medical implantable system, comprising:
defining said structure to be analyzed;
developing a structure model of said structure, developing said structure model comprising generating a part finite element analysis (FEA) model based upon a physical test model and an environmental effect;
performing a physical structure analysis upon said structure, performing said physical structure analysis comprising sending an experimental stiffness curve to a global finite elements analysis (FEA) model, said experimental stiffness curve being based upon a physical test performed on said structure; and
performing an integrating structure analysis based upon said structure model and said physical structure, said integrating structure analysis to generate data for performing at least one of a design, development, simulation, safety evaluation, and manufacturing of said structure based upon said global finite elements analysis (FEA) model generated from said physical test data and said part finite elements analysis (FEA) model.

14. The method described in claim 13, wherein defining said structure to be analyzed further comprises defining at least one of a cardiac pacing lead, a defibrillation lead, a neurological lead, a neurological catheter, a cardiac catheter, and a lead delivery system.

15. The method described in claim 13, further comprising determining a desired physical characteristic for said structure.

16. A system for analyzing a structure for implementation in a medical implantable system, comprising:
means for defining said structure to be analyzed;
means for developing a structure model of said structure, developing said structure model comprising generating a part finite element analysis (FEA) model based upon a physical test model and an environmental effect;
means for performing a physical structure analysis upon said structure, performing said physical structure analysis comprising sending an experimental stiffness curve to a global finite elements analysis (FEA) model, said experimental stiffness curve being based upon a physical test performed on said structure; and
means for performing an integrating structure analysis based upon said structure model and said physical structure, said integrating structure analysis to generate data for performing at least one of a design, development, simulation, safety evaluation, and manufacturing of said structure based upon said global finite elements analysis (FEA) model generated from said physical test data and said part finite elements analysis (FEA) model.

17. The system described in claim 16, further comprising means for determining a desired physical interaction characteristic for said structure.

18. A computer readable program storage device encoded with instructions that, when executed by a computer, performs a method, comprising:
defining said structure to be analyzed;
developing a structure model of said structure;
performing a physical structure analysis upon said structure; and
performing an integrating structure analysis based upon said structure model and said physical structure, said integrating structure analysis to generate data for performing at least one of a design, development, simulation, safety evaluation, and manufacturing of said structure.

19. The computer readable program storage device encoded with instructions that, when executed by a computer, performs the method described in claim 18, the method further comprising implementing said structure into a medical implant system based upon said integrating structure analysis.

20. The computer readable program storage device encoded with instructions that, when executed by a computer, performs the method described in claim 18, further comprising performing a device-anatomy interaction simulation.

21. The computer readable program storage device encoded with instructions that, when executed by a computer, performs the method described in claim 18, wherein performing a physical structure analysis upon said structure further comprises performing a physical stiffness test upon said structure.

22. The computer readable program storage device encoded with instructions that, when executed by a computer, performs the method described in claim 18, wherein defining said structure to be analyzed further comprises defining at least one of a cardiac pacing lead, a defibrillation lead, a neurological lead, a neurological catheter, a cardiac catheter, and a lead delivery system.

23. The computer readable program storage device encoded with instructions that, when executed by a computer, performs the method described in claim 18, wherein developing a structure model of said structure further comprises:
determining a load in a direction of interest for analysis of said structure;
defining a physical stiffness test model based upon said direction of interest of analysis;
evaluating at least one environmental effect upon said structure;
generating a part finite element analysis (FEA) model based upon said physical test model and said environmental effect; and
calibrating a stiffness curve based upon said part finite element analysis (FEA) model.

24. The computer readable program storage device encoded with instructions that, when executed by a computer, performs the method described in claim 23, wherein determining the load in the direction of interest for analysis of said structure further comprises determining one of:
a load of compression force upon said structure;
a load of bending force upon said structure;
a load of torsion force upon said structure;
a load of compression-bending-torsion force upon said structure;
a load of compression-bending force upon said structure; and
a load of three-point bending force upon said structure.

25. The computer readable program storage device encoded with instructions that, when executed by a computer, performs the method described in claim 23, wherein determining at least one environmental effect upon said structure further comprises determining at least one of:
a degradation factor upon said structure;
an aging factor upon said structure;
an erosion factor upon said structure;
a corrosion factor upon said structure;
a temperature effect factor upon said structure; and
a fluid effect factor upon said structure.

26. The computer readable program storage device encoded with instructions that, when executed by a computer, performs the method described in claim 23, wherein generating a part finite element analysis (FEA) model based upon said physical test model and said environmental effect further comprises calibrating a composite structure stiffness for said structure.

27. The computer readable program storage device encoded with instructions that, when executed by a computer, performs the method described in claim 23, wherein calibrating a stiffness curve based upon said part finite element analysis (FEA) model further comprises generating a computed stiffness curve.

28. The computer readable program storage device encoded with instructions that, when executed by a computer, performs the method described in claim 27, wherein performing a physical structure analysis upon said structure further comprises:
performing a physical test of said structure based upon a physical modeling;
generating an experimental stiffness curve based upon said physical test;
comparing said experimental stiffness curve to a target curve from said part finite element analysis (FEA) to generate comparison data;
determining whether said comparison data is inside a predetermined range;
sending said experimental stiffness curve to a global finite elements analysis (FEA) in response to a determination that said comparison data is inside a predetermined range; and
sending said experimental stiffness curve to said part finite elements analysis (FEA) in response to a determination that said comparison data is inside a predetermined range.

29. The computer readable program storage device encoded with instructions that, when executed by a computer, performs the method described in claim 23, wherein performing an integrating structure analysis based upon said structure model and said physical structure
acquiring physical test data based upon said physical structure analysis;
acquiring data from said part finite element analysis (FEA);
generating a global finite element analysis (FEA) model based upon said physical test data and said data from said part finite element analysis (FEA); and
determining a desired physical interaction characteristic for said structure.
